(19) United States
(12) Patent Application Publication
(10) Pub. No.: US 2011/0130666 A1
(43) Pub. Date: Jun. 2, 2011

(54) ENHANCED REPORTING OF PATHOLOGICAL EPISODES

(76) Inventors: Yanting Dong, Shoreview, MN (US); David L. Perschbacher, Coon Rapids, MN (US); Dan Li, Shoreview, MN (US); Deepa Mahajan, Circle Pines, MN (US); F. Roosevelt Gilliam, Jonesboro, AR (US)

(21) Appl. No.: 12/916,843

(22) Filed: Nov. 1, 2010

Related U.S. Application Data
(60) Provisional application No. 61/265,021, filed on Nov. 30, 2009.

(51) Int. Cl. A61B 5/0205; A61B 5/00; A61B 5/0402; A61B 5/0464

(52) U.S. Cl. 600/484; 600/300; 600/515; 600/518; 600/483

(57) ABSTRACT

An apparatus comprises a sensor circuit configured to produce a time-varying physiologic sensor signal of a subject and a pathology detection circuit communicatively coupled to the sensor. The pathology detection circuit is configured to detect a first pathological episode using the sensed physiologic sensor signal, deem that the first pathological episode has ended, detect at least one second pathological episode using the sensed physiologic sensor signal, and indicate the first and second pathological episodes as one pathological episode if the first and second episode are detected within a specified time interval.

### CROSS-REFERENCE TO RELATED APPLICATION

This application claims the benefit of U.S. Provisional Application No. 61/265,021, filed on Nov. 30, 2009, under 35 U.S.C. §119(e), which is incorporated herein by reference in its entirety.

### BACKGROUND

Implantable medical devices (IMDs) include devices designed to be implanted into a patient. Some examples of these devices include cardiac function management (CFM) devices such as implantable pacemakers, implantable cardioverter defibrillators (ICDs), cardiac resynchronization therapy devices (CRTs), and devices that include a combination of such capabilities. The devices can be used to treat patients using electrical or other therapy or to aid a physician or caregiver in patient diagnosis through internal monitoring of a patient's condition. The devices may include one or more electrodes in communication with one or more sense amplifiers to monitor electrical heart activity within a patient, and often include one or more sensors to monitor one or more other internal patient parameters. Other examples of IMDs include implantable diagnostic devices, implantable drug delivery systems, or implantable devices with neural stimulation capability.

Medical devices also include wearable medical devices (WMDs) such as wearable cardioverter defibrillators (WCDs). WCDs are monitors that include surface electrodes. The surface electrodes are arranged to provide one or both of monitoring surface electrocardiograms (ECGs) and delivering cardioverter and defibrillator shock therapy.

Some IMDs detect events by monitoring electrical heart activity signals. In CFM devices, these events can include heart chamber expansions or contractions. By monitoring cardiac signals indicative of expansions or contractions, IMDs can detect abnormally slow heart rate, or bradycardia. Some IMDs detect abnormally rapid heart rate, or tachyarrhythmia. Tachyarrhythmia includes ventricular tachycardia (VT) and supraventricular tachycardia (SVT). Tachyarrhythmia also includes rapid and irregular heart rate, or fibrillation, including ventricular fibrillation (VF). Tachyarrhythmia can also occur in the atria. Examples include atrial fibrillation (AF) and atrial flutter (AFl). Additionally, some IMDs include sensors to monitor physiologic cardiovascular aspects of the patient. IMDs may use such sensors to monitor or measure hemodynamic parameters related to chamber filling and contractions, and other physiological parameters.

IMDs are able to communicate with external devices using wireless communication methods such as radio frequency (RF) or mutual inductance. Some IMDs are able to obtain sampled values of the monitored heart activity signals or values of electrical signals provided by a sensor. The sampled heart activity signals are sometimes referred to as an electrogram. An electrogram can be stored in the IMD and later communicated to an external device where the sampled signals can be displayed for analysis. An electrogram can also be communicated to the external device from the IMD as the heart activity signal is sampled to provide real time electrograms. As with externally obtained electrocardiograms (ECGs), reading, analyzing, and interpreting electrograms can be difficult for a clinician.

### OVERVIEW

This document relates generally to systems, devices, and methods for monitoring cardiac electrophysiological parameters of a patient or subject. Episodes of cardiac arrhythmia or other physiological events are also monitored.

Example 1 includes subject matter (such as an apparatus) comprising a sensor circuit configured to produce a time-varying physiologic sensor signal of a subject and a pathology detection circuit communicatively coupled to the sensor circuit. The pathology detection circuit is configured to detect a first pathological episode using the sensed cardiac signal, deem that the first pathological episode has ended, detect at least one second pathological episode using the physiologic sensor signal, and indicate the first and second pathological episodes as one episode of arrhythmia if the first and second pathological episodes are detected within a specified time interval.

In example 2, the sensor circuit of example 1 can optionally include an implantable cardiac signal sensing circuit configured to produce a sensed cardiac signal representative of cardiac activity of a subject, and the pathology detection circuit is optionally configured to detect a first episode of arrhythmia using the sensed cardiac signal, deem that the first episode of arrhythmia has ended, detect at least one second episode of arrhythmia using the sensed cardiac signal, and indicate the first and second episodes of arrhythmia as one episode of arrhythmia if the first and second episodes are detected within the specified time interval.

In example 3, the pathology detection circuit of any one or more of examples 1 or 2 can optionally be configured to adjust at least one arrhythmia detection parameter when one or more arrhythmia episodes are detected within the specified time interval of a previous arrhythmia episode.

In example 4, when multiple tachyarrhythmia episodes are detected within the specified time interval, the pathology detection circuit of any one or more of examples 1-3 can optionally be configured to reduce an atrial tachyarrhythmia response entry count, increase an atrial tachyarrhythmia response exit count, or reduce a tachyarrhythmia detection rate zone threshold.

In example 5, the pathology detection circuit of any one or more of examples 1-4 can optionally be configured to determine whether the first and second episodes are one episode of arrhythmia by applying an additional detection criterion to the first and second arrhythmia episodes when the second episode is detected within the specified time interval, and indicate the first and second episodes of arrhythmia as one episode of arrhythmia according to the additional detection criterion.

In example 6, the pathology detection circuit of any one or more of examples 1-5 can optionally be configured to obtain, as the additional detection criteria, at least one of similarity of heart rate among detected episodes, an assessment of heart rate stability, or a morphology analysis of the detected arrhythmia.

In example 7, the subject matter of any one or more of examples 1-6 can optionally include a second implantable sensor configured to produce a second sensor signal representative of hemodynamic function of the heart, and the pathology detection circuit is optionally configured to determine whether the first and second episodes are one episode of arrhythmia according to the second sensor signal.

In example 8, the sensor circuit of any one or more of examples 1-7 can optionally include at least one of: an implantable respiration sensor configured to produce a sensed respiration signal representative of respiration activity of the subject and the first and second pathological episodes include episodes of fast respiration rate, an implantable blood pressure sensor configured to produce a sensed pressure signal representative of blood pressure of the subject and the first and second pathological episodes include episodes of one or more low blood pressure and high blood pressure, an implantable blood gas sensor configured to produce a sensor signal associated with changes in the fluid oxygen saturation of blood and the first and second pathological episodes include episodes of a low level of oxygen saturation in blood, an implantable chemical sensor configured to produce a sensor signal associated with changes in the blood pH, and the first and second pathological episodes include episodes of a change in blood pH that exceeds a specified change value, or a heart sound sensor configured to produce a heart sound signal associated with mechanical activity of a patient's heart and the first and second pathological episodes include episodes of a change in a measured heart sound parameter that exceeds a specified change value.

In example 9, the pathology detection circuit of any one or more of examples 1-8 can optionally be configured to indicate the first pathological episode and the second pathological episode as one pathological episode according to additional detection criteria. The additional detection criteria optionally includes at least one of similarity of morphology of the physiologic sensor signal during the detected episodes, similarity of device-determined posture of the subject during the episodes, or similarity of device-determined activity level of the subject during the episodes.

In example 10, the pathology detection circuit of any one or more of examples 1-9 can optionally include a pathological episode counter, and the pathology detection circuit is configured to update the pathological episode counter upon detecting the first pathological episode, maintain an episode count of the pathological episode counter when one or more later pathological episodes are detected within the specified time interval of a previous pathological episode, and update the pathological episode counter upon detecting a later pathological episode after the time interval expires.

Example 11 can include, or can optionally be combined with the subject matter of any one or more of examples 1-10 to include, subject matter (such as a system) comprising an IMD and an external device. The IMD comprises an implantable sensor configured to produce a time-varying physiologic sensor signal of a subject, and a pathology detection circuit communicatively coupled to the sensor and configured to detect a first pathological episode using the physiologic sensor signal, deem that the first pathological episode has ended, detect at least one second pathological episode using the physiologic sensor signal, and indicate the first and second pathological episodes as one pathological episode if the first and second episodes are detected within a specified time interval. The IMD also includes a communication circuit communicatively coupled to the pathology detection circuit and configured to communicate information wirelessly with an external device. The pathology detection circuit is configured to communicate an indication of the one pathological episode to the external device. The external device comprises a user interface, a communication circuit, and a processor communicatively coupled to the user interface and the communication circuit and configured to display the first and second pathological episodes as one pathological episode according to an indication received from the IMD.

In example 12, the processor of example 11 can optionally be configured to display the indicated one pathological episode as multiple pathological episodes according to input received via a user interface at the external device, display the multiple pathological episodes using multiple segments of the physiologic sensor signal according to input received via the user interface, and display a statistic related to a pathological episode in association with a segment of the physiologic sensor signal according to information received from the IMD.

In example 13, the pathology detection circuit of any one or more of examples 11 and 12 can optionally be configured to detect the first pathological episode using a first segment of the physiologic sensor signal, detect the second pathologic episode using a second segment of the physiologic sensor signal, transmit representations of the first and second segments to the external device. The processor is optionally configured to display a statistic related to the detected pathology as one episode, and display a pathological statistic related to each of the first and second segments.

Example 14 can include, or can optionally be combined with the subject matter of any one or combination of Examples 1-13 to include, subject matter (such as a method, a means for performing acts, or a machine-readable medium including instructions that, when performed by a machine, cause the machine to perform acts) comprising detecting a first pathological episode of a subject with a device using a sensed time-varying physiologic sensor signal, deeming that the first pathological episode has ended, detecting at least one second pathological episode with the device, and deeming, with the device, that the first and second pathological episodes are one pathological episode if the first and second episodes are detected within a specified time interval.

In example 15, the detecting a first pathological episode of example 14 can optionally include detecting a first episode of arrhythmia with an IMD, the deeming that the first pathological episode has ended optionally includes deeming that the first episode of arrhythmia has ended, the detecting at least one second pathological episode optionally includes detecting at least one second episode of arrhythmia with the IMD, and the deeming that the first and second pathological episodes are one episode optionally includes deeming that the first and second episodes of arrhythmia are one episode of arrhythmia if the first and second episodes are detected within the specified time interval.

In example 16, the subject matter of any one or more of examples 14 and 15 can optionally include adjusting at least one arrhythmia detection parameter if multiple arrhythmia episodes are detected within the specified time interval of a previous arrhythmia episode.

In example 17, the adjusting at least one arrhythmia detection parameter of example 16 can optionally include at least one of reducing an atrial tachyarrhythmia response entry count, increasing an atrial tachyarrhythmia response exit count, or reducing a tachyarrhythmia rate detection zone threshold.

In example 18, the subject matter of any one or more examples 14-17 can optionally include determining whether the first and second episodes are one episode of arrhythmia by applying an additional detection criterion to the first and second arrhythmia episodes when the second episode is detected within the specified time interval. The indicating the first and second episodes of arrhythmia as one episode of arrhythmia optionally includes indicating the first and second episodes of arrhythmia as one episode of arrhythmia according to the additional detection criteria.

In example 19, the additional detection criteria of example 18 can optionally include at least one of an assessment of heart rhythm stability, a morphology analysis of the detected arrhythmia, or an assessment of hemodynamic stability.

In example 20, the detecting the first and second pathological episodes with the IMD of any one or more of examples 14-19 can optionally include detecting at least one of first and second episodes of fast respiration rate, first and second episodes of one or more of low blood pressure and high blood pressure, first and second episodes of low levels of oxygen saturation in blood, first and second episodes of a change in blood pH that exceeds a specified change value, or first and second episodes of a change in a measured heart sound parameter that exceeds a specified change value.

In example 21, the deeming that the first and second pathological episodes are one pathological episode of any one or more of example 18-20 can optionally include deeming that the first and second pathological episodes are one pathological episode according to additional detection criteria that optionally includes at least one of similarity of morphology of the physiologic sensor signal during the detected first and second episodes, similarity of device-determined posture of the subject during the first and second episodes, or similarity of device-determined activity level of the subject during the first and second episodes.

In example 22, the subject matter of any one or more of examples 14-21 can optionally include updating a pathological episode counter upon detecting the first pathological episode, maintaining an episode count when one or more later pathological episodes are detected within the specified time interval of a previous pathological episode, and updating the arrhythmia episode counter upon detecting a later pathological episode after the specified time interval expires.

In example 23, the indicating the first and second pathological episodes as one pathological episode of any one or more of examples 14-22 can optionally include transmitting the indication from an IMD to an external device, and displaying the first and second pathological episodes as one pathological episode or as multiple pathological episodes according to input received via a user interface at the external device.

In example 24, the subject matter of any one or more of examples 14-23 can optionally include displaying the multiple pathological episodes using multiple segments of the physiologic sensor signal when prompted to do so via the user interface, and displaying statistics related to each pathological episode in association with the associated signal segment.

In example 25, the displaying statistics of example 24 can optionally include displaying at least one of a sensed cardiac depolarization rate during each pathological episode, a duration of each pathological episode, or a signal morphology of the physiologic sensor signal during each pathological episode.

This section is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of an example of portions of a system that includes an IMD.

FIG. 2 is a flow diagram of an example of a method of providing enhanced reporting of pathological episodes using a medical device.

FIGS. 3A and 3B show conceptual examples of reporting pathological episodes.

FIG. 4 is a block diagram of portions of an IMD that provides for enhanced reporting of pathological episodes.

FIG. 5 is a block diagram of portions of a system that provides for enhanced reporting of pathological episodes.

### DETAILED DESCRIPTION

An medical device (e.g., an IMD or WMD) may include one or more of the features, structures, methods, or combinations thereof described herein. For example, a cardiac monitor or a cardiac stimulator may be implemented to include one or more of the advantageous features or processes described below. It is intended that such a monitor, stimulator, or other implantable or partially implantable device need not include all of the features described herein, but may be implemented to include selected features that provide for unique structures or functionality. Such a device may be implemented to provide a variety of therapeutic or diagnostic functions.

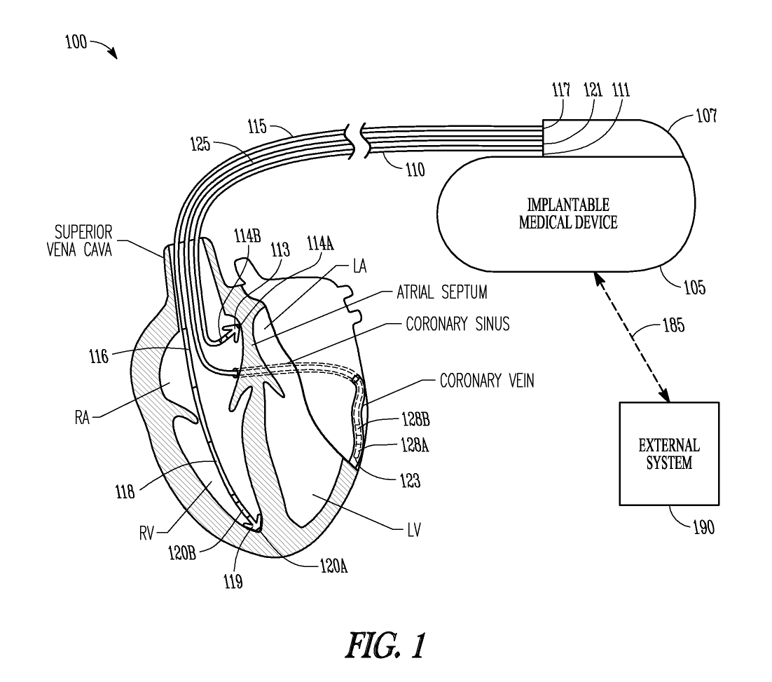

FIG. 1 is an illustration of portions of a system 100 that uses an IMD 105. Examples of the IMD 105 include, without limitation, a pacemaker, a cardioverter, a defibrillator, a cardiac resynchronization therapy (CRT) device, and other cardiac monitoring and therapy delivery devices, including cardiac devices that include or work in coordination with one or more neuro-stimulating devices, drugs, drug delivery systems, or other therapies. As one example, the system 100 shown can be used to detect and treat a cardiac arrhythmia such as tachyarrhythmia. The IMD 105 typically includes an electronics unit coupled by one or more cardiac leads 110, 115, 125, to a heart of a patient or subject. The electronics unit of the IMD 105 typically includes components that are enclosed in a hermetically-sealed housing or “can.” System 100 also typically includes an IMD programmer or other external system 190 that communicates one or more wireless signals 185 with the IMD 105, such as by using radio frequency (RF) or one or more other telemetry signals.

The example shown includes right atrial (RA) lead 110 having a proximal end 111 and a distal end 113. Proximal end 111 is coupled to a header connector 107 of the IMD 105. Distal end 113 is configured for placement in the RA in or near the atrial septum. RA lead 110 may include a pair of bipolar electrodes, such as an RA tip electrode 114A and an RA ring electrode 114B. RA electrodes 114A and 114B are incorporated into the lead body at distal end 113 for placement in or near the atrial septum, and are each electrically coupled to IMD 105 through a conductor extending within the lead body. The RA lead is shown placed in or near the atrial septum, but the RA lead may be placed in the atrial appendage.

The example shown also includes right ventricular (RV) lead 115 having a proximal end 117 and a distal end 119. Proximal end 117 is coupled to header connector 107. Distal end 119 is configured for placement in the RV. RV lead 115 may include one or more of a proximal defibrillation electrode 116, a distal defibrillation electrode 118, an RV tip electrode 120A, and an RV ring electrode 120B. Defibrillation electrode 116 is incorporated into the lead body in a location suitable for supraventricular placement in the RA or the superior vena cava. Defibrillation electrode 118 is incorporated into the lead body near distal end 119 for placement in the RV. RV electrodes 120A and 120B may form a bipolar electrode pair and are incorporated into the lead body at distal end 119. Electrodes 116, 118, 120A, and 120B are each electrically coupled to IMD 105 through a conductor extending within the lead body. Proximal defibrillation electrode 116, distal defibrillation electrode 118, and/or an electrode formed on the can of IMD 105 allow for delivery of cardioversion/defibrillation pulses to the heart.

RV tip electrode 120A, RV ring electrode 120B, and/or an electrode formed on the can of IMD 105 allow for sensing an RV electrogram indicative of RV depolarizations and delivering RV pacing pulses. RA tip electrode 114A, RA ring electrode 114B, and/or an electrode formed on the can of IMD 105 allow for sensing an RA electrogram indicative of RA depolarizations and delivering RA pacing pulses. Sensing and pacing allows the IMD 105 to adjust timing of the heart chamber contractions. In some device examples, IMD 105 can adjust the timing of ventricular contractions with respect to the timing of atrial contractions delay by sensing a contraction in the RA and pacing the RV at the desired atrial-ventricular (AV) delay time.

Also shown is a left ventricular (LV) lead 125. LV lead 125 is a coronary pacing and/or sensing lead that includes an elongate lead body having a proximal end 121 and a distal end 123. Proximal end 121 is coupled to header connector 107. Distal end 123 is configured for placement or insertion in the coronary vein. LV lead 125 may include an LV ring or tip electrode 128A and an LV ring electrode 128B. The distal portion of LV lead 125 is configured for placement in the coronary sinus and coronary vein such that LV electrodes 128A and 128B are placed in the coronary vein. LV electrodes 128A and 128B may form a bipolar electrode pair and are incorporated into the lead body at distal end 123 and each electrically coupled to IMD 105 through a conductor extending within the lead body. LV tip electrode 128A, LV ring electrode 128B, and/or an electrode formed on the can of IMD 105 allow for sensing an LV electrogram indicative of LV depolarizations and delivering LV pacing pulses.

Other forms of electrodes include meshes and patches, which may be applied to one or more portions of heart, or which may be implanted in one or more other areas of the body to help “steer” electrical current produced by IMD 105 in FIG. 1. The IMDs may be configured with a variety of electrode arrangements, including transvenous, endocardial, or epicardial electrodes (e.g., intrathoracic electrodes), or subcutaneous, non-intrathoracic electrodes, such as can, header, or indifferent electrodes, or subcutaneous array or lead electrodes (e.g., non-intrathoracic electrodes). Monitoring of electrical signals related to cardiac activity may provide early, if not immediate, diagnosis of cardiac disease.

An IMD may include one or more sensors. The sensors provide a time-varying electrical signal that is related to physiologic cardiovascular events of a subject. A non-exhaustive list of examples of such sensors include a cardiac signal sensing circuit, an intracardiac impedance sensing circuit, a transthoracic impedance sensing circuit, a blood pressure sensor, a blood gas sensor, a chemical sensor, a heart sound sensor, a posture sensor, and an activity sensor. In some examples, the IMD communicates with a sensor external to the IMD. The signals provided by the sensors may be used to detect a pathological event or episode that a patient or subject is experiencing or has experienced.

For example, the IMD may be able to detect an arrhythmic event from a cardiac signal sensed using any of the electrodes described. The cardiac signal is representative of cardiac activity of a subject or patient. When a pathological episode such as an episode of arrhythmia is detected, the IMD may begin recording the cardiac signal (e.g., as an electrogram). The recorded cardiac signal may then be communicated to an external device. In general, every arrhythmic episode detected by an IMD is treated as unique. However, a series of episodes of arrhythmia experienced by a subject may actually be related. Physiological information useful to a clinician may be missed when a device indicates that detected pathological episodes are unique.

For example, some patients with AF may have hundreds of atrial tachyarrhythmia response (ATR) episodes in a single day. However, from a clinical perspective, this may be one episode of AF, whereas a device (e.g., an IMD or an external device) may report the AF as many episodes. This may happen for several reasons. The multiple episode reporting may be due to the detection entry and exit criteria programmed into an IMD, due to under-sensing of signals in an atrium, or due to device suspension of arrhythmia detection during a delivery of therapy or suspension of detection during a device follow-up procedure. This same scenario may occur for ventricular tachyarrhythmia. The device may report or indicate several different episodes of ventricular tachyarrhythmia even though this may be one episode from a clinical perspective.

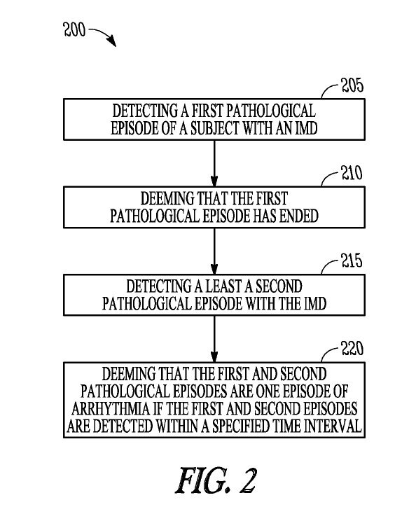

FIG. 2 is a flow diagram of an example of a method 200 of providing enhanced reporting of pathological episodes using a medical device. At block 205, a first pathological episode is detected with an IMD. The episode is detected using a sensed time-varying physiologic sensor signal.

In some examples, the first pathological episode is an episode of tachyarrhythmia such as, among other things, VT, VF, SVT, sinus tachycardia (ST), AF, or AFl. In certain examples, the IMD senses cardiac depolarization signals and detects tachyarrhythmia by detecting a depolarization rate that exceeds a tachyarrhythmia detection rate threshold and that the depolarization rate is sustained for a period of time.

At block 210, the IMD deems that the first pathological episode has ended. In tachyarrhythmia example, the episode is deemed to have ended when the IMD detects that the depolarization is below the detection rate threshold for a specified period of time or for a specified number of cardiac cycles.

At block 215, at least one second pathological episode is detected with the IMD. Again using the episode of tachyarrhythmia as an example, there may be several (even hundreds) tachyarrhythmia episodes that are detected and deemed to have ended. At block 220, the first and second pathological episodes are indicated as one pathological episode if the first and second pathological episodes are detected within a specified time interval.

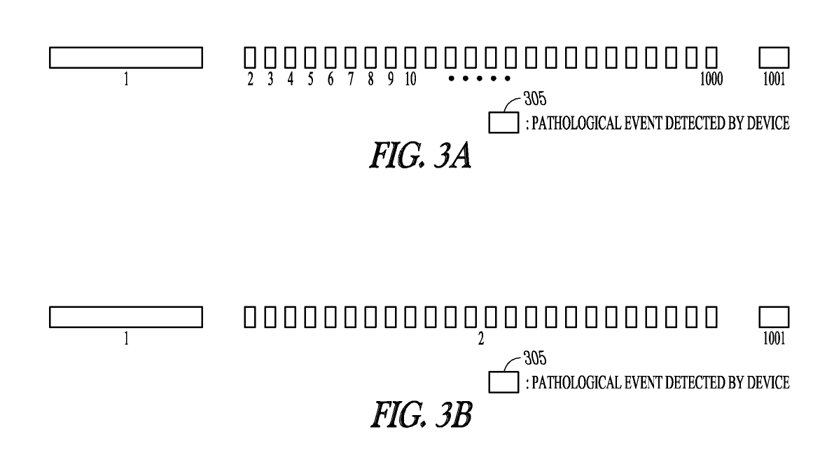

FIGS. 3A and 3B show examples of reporting pathological episodes. In the Figures, the box 305 represents a pathological event or episode. In FIG. 3A, each detected episode is indicated separately as episodes 1 through 1001. In FIG. 3B, episodes 2 through 1000 in FIG. 3A occur within the specified time interval, so those episodes are grouped together into episode 2. Therefore, only three pathological episodes are indicated in FIG. 3B. In some examples, the specified time interval is a period of time when one episode ends and the next episode begins (e.g., each episodes 2 through 1000 each occur within a specified time from the previous episode). Episode 2 is not grouped with episode 1 because episode 2 did not occur within the specified time period of episode 1. The grouping of the episodes may be useful to the clinician in interpreting and analyzing the detected episodes. In certain examples, the specified time period is a window of time that is begun after the first episode is detected. Sub-episodes that fall within the window of time are grouped together as one episode. However, the size of the window may limit the number of sub-episodes that can be indicated as one episode. Returning to FIG. 2, as is described below, additional criteria is optionally applied to the first and second episodes to determine if they are indeed one pathological episode.

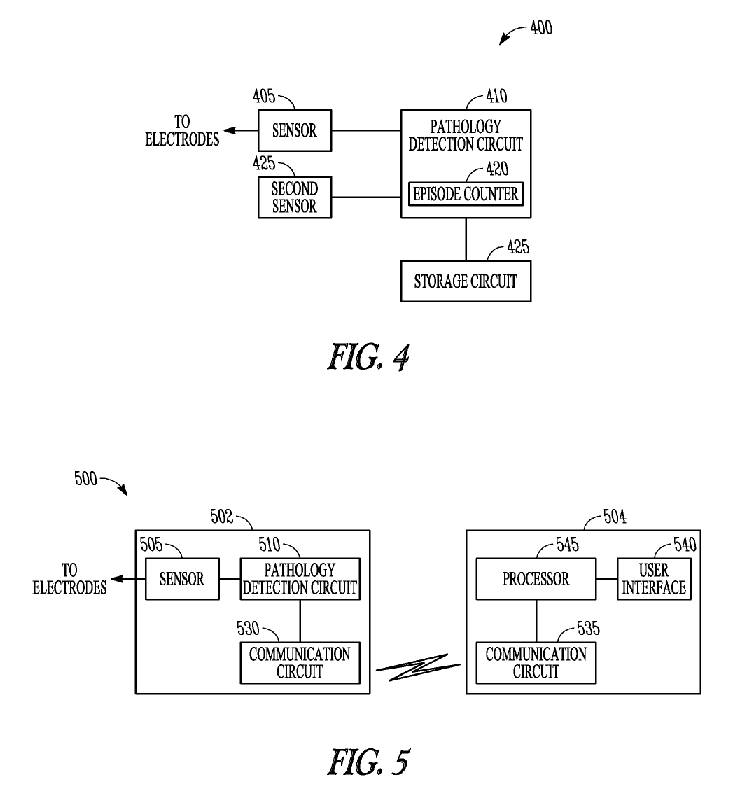

FIG. 4 is a block diagram of portions of an IMD 400 that provides for enhanced reporting of pathological episodes. The IMD 400 includes an implantable sensor 405 and a pathology detection circuit 410 communicatively coupled to the sensor 405. The communicative coupling provides for exchange of electrical signals between the sensor 405 and the pathology detection circuit 410 even though there may be intervening circuitry. For example, signal sampling circuitry may present digitized values of an electrical signal produced by the sensor 405 to the pathology detection circuit 410.

In some examples, the pathology detection circuit 410 includes a processor and performs one or more detection algorithms that are embodied in instructions in software or firmware that are performable by the processor. Such a processor may include a microprocessor, a digital signal processor (DSP), or application specific integrated circuit (ASIC).

The sensor 405 produces a sensed time-varying physiologic sensor signal that is related to a physiologic condition of the subject. The pathology detection circuit 410 detects a first pathological episode using the sensor signal.

For example, the sensor 405 may include an implantable cardiac signal sensing circuit that produces a sensed cardiac signal representative of cardiac activity of a subject. In some examples, the sensed cardiac signal is representative of cardiac depolarization events. The cardiac signal sensing circuit senses the signals when it is electrically coupled to electrodes. The pathology detection circuit 410 detects a first episode of tachyarrhythmia using the sensed cardiac signal. In some examples, the pathology detection circuit 410 detects tachyarrhythmia using the sensed cardiac signal, such as by detecting a depolarization rate that exceeds a tachyarrhythmia detection rate threshold or is less than a depolarization interval threshold. In certain examples, the pathology detection circuit 410 detects tachyarrhythmia when the rate or interval is sustained for a specified duration of time or specified number of cardiac cycles. This can be referred to as an entry count for declaring a tachyarrhythmia. The time interval or the number of cardiac cycles may be specified by programming a value into the pathology detection circuit 410 or by setting the value in firmware or hardware.

As an illustrative example, the specified number of cardiac cycles or beats for declaring tachyarrhythmia can be set to 8. The pathology detection circuit 410 may include a beat counter to track the number of beats that satisfies the detection rate or interval. If a fast beat is detected having an interval less than a detection threshold interval (e.g., an interval corresponding to a rate of 170 beats per minute (bpm) or 350 ms), the counter is incremented. Tachyarrhythmia will be declared if 8 fast beats are detected. The beats can be required to be consecutive or to satisfy an X out of Y requirement, such as 8 beats out of 10 beats being fast beats.

In certain examples, the pathology detection circuit 410 detects arrhythmia using an assessment of heart rhythm stability when a subject experiences a sudden increase in heart rate. Examples of methods and systems to detect arrhythmia and assess the stability of the rhythms are found in Gilkerson et al., U.S. Pat. No. 6,493,579, entitled “System and Method for Detection Enhancement Programming,” filed Aug. 20, 1999, which is incorporated herein by reference in its entirety.

The pathology detection circuit 410 also deems when the first pathological episode has ended. For the tachyarrhythmia example, if 8 long intervals or slow beats are detected (sometimes referred to as an exit count), then the beat counter decrements to zero and the episode is deemed to have ended. In certain examples, the IMD 400 includes a storage circuit 415 (e.g., a memory) integral to or communicatively coupled to the pathology detection circuit 410, and timestamps are stored to mark the duration of an episode.

According to some examples, the sensor 405 includes an implantable respiration sensor configured to produce a sensed respiration signal representative of respiration activity of the subject. An example of an implantable respiration sensor is a transthoracic impedance sensor to measure minute respiration volume. An approach to measuring transthoracic impedance is described in Hartley et al., U.S. Pat. No. 6,076,015 “Rate Adaptive Cardiac Rhythm Management Device Using Transthoracic Impedance,” filed Feb. 27, 1998, which is incorporated herein by reference. The pathology detection circuit 410 detects a first pathological episode that includes an episode of fast respiration rate, such as when the rate exceeds a resting rate by a specified threshold rate or a specified percentage of the resting rate. The pathology detection circuit 410 detects an end of the episode when the respiration rate drops below the same or a different specified threshold rate.

In some examples, the sensor 405 includes an implantable blood pressure sensor configured to produce a sensed pressure signal representative of blood pressure of the subject. In an example, a left ventricular pressure sensor is implanted in a coronary vessel to determine left ventricle pressure by direct measurement of coronary vessel pressure. A description of systems and methods that use such an implantable pressure sensor is found in Salo et al., U.S. Pat. No. 6,666,826, entitled “Method and Apparatus for Measuring Left Ventricular Pressure,” filed Jan. 4, 2002, which is incorporated herein by reference. Other cardiac pressure sensors examples include a right ventricle (RV) chamber pressure sensor, a left atrial chamber pressure sensor, and a pulmonary arterial (PA) pressure sensor. PA pressure includes the pressure within a pulmonary artery due to blood leaving the right ventricle through the pulmonary valve and going to the lungs. The pathology detection circuit 410 detects a first pathological episode that includes an episode of low blood pressure or high blood pressure. The pathology detection circuit 410 detects an end of the pathological episode when the blood pressure returns to a normal range.

In some examples, the sensor 405 includes an implantable blood gas sensor. An example of a blood gas sensor is an implantable oxygen saturation sensor. An oxygen saturation sensor produces an electrical sensor signal associated with changes in the fluid oxygen saturation. Such changes may occur in association with the heart's mechanical activity, contractility, or blood flow. The pathology detection circuit 410 detects a first pathological episode that includes an episode of low levels of oxygen saturation in the blood of the subject. The pathology detection circuit 410 detects an end of the pathological episode when the oxygen saturation returns to a target value or range.

In some examples, the sensor 405 includes an implantable chemical sensor. Illustrative examples include a blood electrolyte sensor, such as to detect one or more of potassium (K), sodium (Na) calcium (Ca), glucose, or creatinine. In some examples, a blood chemical sensor detects changes in blood pH. An example of an approach to providing a chemical sensor in a coronary sinus is found in Kane et al., U.S. patent application Ser. No. 11/383,933, entitled, “Implantable Medical Device with Chemical Sensor and Related Methods, filed May 17, 2006, which is incorporated herein by reference. The sensor 405 is configured to produce a sensor signal associated with changes in the blood electrolytes or pH. In certain examples, the pathology detection circuit 410 detects a first pathological episode that includes an episode of a change in blood pH that exceeds a specified change value.

In some examples, the sensor 405 includes an implantable heart sound sensor configured to produce a heart sound signal associated with mechanical activity of a patient's heart. Heart sounds are associated with mechanical vibrations from activity of a patient's heart and the flow of blood through the heart. Heart sounds recur with each cardiac cycle and are separated and classified according to the activity associated with the vibration. The first heart sound (S1) is the vibrational sound made by the heart during tensing of the mitral valve. The second heart sound (S2) marks the beginning of diastole. The third heart sound (S3) and fourth heart sound (S4) are related to filling pressures of the left ventricle during diastole.

A heart sound sensor produces an electrical signal which is representative of mechanical activity of a patient's heart. An approach for monitoring heart sounds is found in Siejko et al., U.S. Patent Application Publ. No. 2004/0127792, entitled “Method and Apparatus for Monitoring of Diastolic Hemodynamics,” filed Dec. 30, 2002, which is incorporated herein by reference in its entirety.

In certain examples, the pathology detection circuit 410 detects a first pathological episode that includes a change in a measured heart sound parameter that exceeds a specified change value. The pathology detection circuit 410 detects an end of the pathological episode when the heart sound parameter returns to a target value or range.

After detecting the first pathological episode and that this first episode has ended, the pathology detection circuit is configured to detect at least one second pathological episode using the physiologic sensor signal. In some examples, the first pathological episode is detected during a first segment of the sensed physiologic signal. The pathology detection circuit 410 may then detect at least one second episode of arrhythmia during a second segment of the sensed physiologic signal.

The pathology detection circuit 410 indicates the first and second pathological episodes as one pathological episode if the first and second episodes are detected within a specified time interval. The pathology detection circuit 410 may include a timer circuit to time the interval and determine when the interval expires by comparison to a programmable time interval value. In some examples, the pathology detection circuit 410 detects more than one type of pathology. The pathology detection circuit 410 may use different specified time intervals to group different types of pathological episodes. For example, if the pathology detection circuit 410 detects different types of arrhythmias, the pathology detection circuit 410 may use different specified time intervals to group atrial arrhythmias and to group ventricular arrhythmias. In certain examples, the grouping of episodes is enabled only for atrial events and not for ventricular events, and vice versa.

In some examples, the time interval value used in the comparison is adaptable based on the type of pathology detected. The pathology detection circuit 410 may set the value according to the pathology detected. For instance, the pathology detection circuit 410 may set the value according to a heart rate of a detected arrhythmia. The pathology detection circuit 410 may determine the value using a look-up table referenced by one or both of arrhythmia type and heart rate.

As described previously, the pathology detection circuit 410 may detect many subsequent pathological episodes. All the episodes are indicated to be the same episode if a subsequent or later episode follows a previous or earlier episode within the specified time interval.

In some examples, the indication is an episode count. The pathology detection circuit 410 includes a pathological episode counter 420, and the pathology detection circuit 410 updates the pathological episode counter 420 upon detecting the first pathological episode. When the next pathological episode is detected, the episode count of the pathological episode counter 420 is maintained (e.g., not changed) when the pathological episodes are detected within the specified time duration. The pathological episode counter 420 is updated (e.g., increased) when the next pathological episode is detected after the specified time duration expires.

According to some examples, the pathology detection circuit 410 changes at least one pathology detection parameter when multiple pathological episodes are detected within the specified time interval. In certain examples, the pathology detection circuit 410 may change a parameter to make the device more sensitive to detection of pathological episodes. For instance, the parameter may be a detection threshold value of the physiologic sensor signal, and the value is changed so that pathological episode detection is more inclusive.

If the detected pathology is arrhythmia, the pathology detection circuit 410 may change at least one arrhythmia detection parameter or therapy parameter when multiple arrhythmia episodes are detected within the specified time interval. For instance, the parameter may be an entry count such as an atrial tachyarrhythmia response (ATR) entry count. The pathology detection circuit 410 may change the entry count to a smaller number (e.g., reduce the count from 10 to 8 consecutive fast beats) or a lower number of non-consecutive beats (e.g., from 8 fast beats out of 10 beats to 6 fast beats out of 10 beats). In another example, the parameter may be a tachyarrhythmia detection rate zone threshold. The pathology detection circuit 410 may reduce the rate zone threshold (e.g., from 180 bpm to 170 bpm) to make tachyarrhythmia detection more inclusive.

In certain examples, the pathology detection circuit 410 may change a parameter to make redetection of subsequent episodes less sensitive. This makes it more likely that subsequent arrhythmia episodes will be seen as part of an earlier detected episode. For instance, the pathology detection circuit 410 may increase an atrial tachyarrhythmia response exit count (e.g., increase the exit count from 8 slow beats to 10 slow beats).

In some examples, the pathology detection circuit 410 changes the pathology detection parameters when the first pathological episode is detected. The pathology detection circuit 410 returns the parameters to their original (e.g., default or programmed) values after a time interval. The time interval may be the specified time interval used to detect separate pathological episodes, or may be a different (e.g., longer) time interval.

Other criteria may be used to determine whether multiple pathological episodes should be reported as the same episode. In some examples, the pathology detection circuit 410 applies an additional detection criterion to first and second detected pathological episodes when the second episode is detected within the specified time interval, and indicates the first and second pathological episodes as one pathological episode or multiple pathological episodes according to the additional detection criteria.

According to some examples, the additional detection criteria include a determination of the similarity of morphology of the physiologic sensor signal used to detect the episodes. If the multiple pathological episodes have a similar morphology it is more likely that the multiple episodes may be viewed as the same pathological episode.

For instance, if the pathological episode is arrhythmia, the pathology detection circuit 410 uses morphology similarity to a template to determine whether to group detected episodes of arrhythmia. The pathology detection circuit 410 compares the morphology of a segment of the sensed cardiac signal to a morphology template stored in the storage circuit 415. In some examples, the morphology of a sensed cardiac depolarization is compared to a template of a known normal or abnormal depolarization morphology (such as NSR, VT, or SVT) stored in the storage circuit 415. For example, a template can be created for a patient using a CRM by providing electrical energy pulses to the supra-ventricular region of the patient's heart. The resulting cardiac complexes are then sensed and used to create a template for use in a morphology-based cardiac signal classification algorithm. Systems and methods of creating templates for a morphology-based algorithm are described in Hsu, U.S. Pat. No. 6,889,081, entitled “Classification of Supra-ventricular and Ventricular Cardiac Rhythms Using Cross Channel Timing Algorithm,” filed Jul. 23, 2002, which is incorporated herein by reference in its entirety. The comparison to the template or templates may include calculating a score of similarity to the template. Episodes with similar scores (e.g., segments having scores within a specified range of scores) are grouped together as one episode of the detected arrhythmia.

In some examples, the additional detection criteria include an assessment of heart rhythm stability when a subject experiences a sudden increase in heart rate (e.g., ventricular rate). In some examples, the additional detection criteria include a detected similarity of heart rate among detected arrhythmia episodes. For instance, the pathology detection circuit 410 may group two sub-episodes of tachyarrhythmia together as one episode if the detected rate of the two sub-episodes are have the same rate (e.g., 180 bpm) or have rates within a specified range (e.g., 180-185 bpm). In some examples, the pathology detection circuit 410 may group two or more sub-episodes of arrhythmia together as one episode if there is a similarity in timing patterns between atrial to ventricular (AV) depolarizations.

In some examples, the additional criteria include using an event or events sensed in one heart chamber to group events sensed in another heart chamber as one episode. For instance, the pathology detection circuit 410 may determine that an arrhythmia sensed in the right atrium is AF. In response to the detected AF, the pathology detection circuit 410 may group together all episodes of arrhythmia detected in one or both ventricular chambers during the AF as one episode of ventricular arrhythmia.

According to some examples, the additional detection criteria include similarity of IMD-determined posture of the subject during the episodes. Multiple pathological episodes that occur within the specified time interval while the patient is in a same determined posture are more likely to be part of one pathological episode. Posture can be determined from a posture sensor included in the IMD 400, such as a two-axis accelerometer, or posture can be deduced, such as from time of day according to a circadian cycle. A clock circuit may be included in the IMD 400 used to determine time of day.

In some examples, the additional detection criteria include similarity of IMD-determined activity of the subject during the episodes. It may be more likely that multiple pathological episodes should be grouped into one pathological episode when the episodes occur within the specified time interval and while the patient has a similar level of activity. In certain examples, the IMD 400 includes an accelerometer communicatively coupled to the pathology detection circuit 410 to determine activity of the subject.

According to some examples, the IMD 400 includes a second implantable sensor 425 communicatively coupled to the pathology detection circuit 410. The second implantable sensor provides additional information to determine whether multiple pathological episodes should be reported as the same episode. The second implantable sensor 425 produces a second sensor signal representative of hemodynamic function of the heart. Hemodynamic function relates to the efficacy of the mechanical function of the heart (e.g., the contractility of the heart). It should be noted this is different from sensing electrical intrinsic cardiac signals which are the action potentials that propagate through the heart's electrical conduction system. The physiologic sensor 405 and the second sensor 425 are typically not the same type of sensor so that additional information is provided to the pathology detection circuit 410. The pathology detection circuit 410 indicates the first and second pathological episodes as one pathological episode or multiple pathological episodes according to information provided by the second sensor signal.

In some examples, the electrical sensor signal is indicative of cardiac output during the pathological event. This may include an electrical signal provided by an implantable cardiac blood pressure sensor. The pathology detection circuit 410 indicates detected pathological episodes as one pathological episode or multiple pathological episodes according to the similarity in the behavior of the indicated blood pressure during the pathological episodes detected using the physiologic sensor signal. Another sensor that provides an electrical sensor signal indicative of cardiac output is a blood flow sensor.

In some examples, the electrical sensor signal is indirectly indicative of cardiac output during the pathological event. Examples of sensors that provide an electrical signal indirectly indicative of hemodynamic function include, among other things, an intracardiac impedance sensor, a transthoracic impedance sensor, a heart sound sensor, a temperature sensor, and a chemical sensor.

Electrodes placed within a chamber of the heart provide a signal of intracardiac impedance versus time. The electrodes may be placed in a right ventricle of the heart and the measured intracardiac impedance waveform can be signal processed to obtain a measure of the time interval beginning with a paced or spontaneous QRS complex (systole marker) and ending with a point where the impedance signal crosses the zero axis in the positive direction following the QRS complex. The resulting time interval is inversely proportional to the contractility of the heart. Systems and methods to measure intracardiac impedance are described in Citak et al., U.S. Pat. No. 4,773,401, entitled “Physiologic Control of Pacemaker Rate Using Pre-Ejection Interval as the Controlling Parameter,” filed Aug. 21, 1987, which is incorporated herein by reference in its entirety. The pathology detection circuit 410 indicates detected pathological episodes as one pathological episode or multiple pathological episodes according to the similarity in measurements of impedance during the episodes.

Hemodynamic function of the heart can also be observed or assessed by monitoring heart sounds. A change in heart chamber contractility can be measured using a heart sound sensor. The pathology detection circuit 410 indicates detected pathological episodes as one pathological episode or multiple pathological episodes according to similarity in heart sound measurements determined during the episodes.

According to some examples, the pathology detection circuit 410 may store information about the episodes in the storage circuit 415. When detecting the pathological episodes, a first detected pathological episode is evident during a first segment of the sensed physiologic sensor signal, a second pathological episode is evident during a second segment of the physiologic sensor signal, and so on. The pathology detection circuit 410 detects the first pathological episode using the first segment and detects the second pathological episode using the second segment of the physiologic sensor signal. In some examples, if the pathology detection circuit 410 deems that the episodes should be grouped together as one episode, the pathology detection circuit 410 only stores information related to the first episode. For instance, the pathology detection circuit 410 may only store a marker that is associated with only the first pathological episode, or store a portion of only the first segment of the physiologic sensor signal, or store both the marker and the segment from only the first pathological episode.

A marker can be a fiducial marker to indicate some event such as the type of pathological episode. The marker can be stored in association with a timestamp of the occurrence. Segments of the physiologic sensor signal can be sampled to obtain a signal segment (e.g., an electrogram). The sampled values are a presentation of a segment of the sensed physiologic signal that can be stored. The segments may be a representation of a continuous signal, or the segments may be formed from sampling the signal at non-continuous specified times. In some examples, the marker is stored in association with the segment to provide annotated signal segments.

In some examples, upon detecting the pathological episodes, the pathology detection circuit 410 stores at least a portion of both the first physiologic sensor signal segment and the second (or subsequent) signal segments. The subsequent episodes of a group may be less important than the first episode of the group, so the subsequent segments may be stored with a different compression scheme from the first segment. In certain examples, the pathology detection circuit 410 stores episodes of a group that occur after the first episode using a using a data compression scheme that results in less accurate reproduction of the second segment than the first segment (e.g., the compression uses less memory to store the device to save memory space). In certain examples, the first segment is stored using a lossless compression scheme, and the subsequent segments are stored using a lossy compression scheme.

In some examples, the pathology detection circuit 410 stores one or both of markers and a portion of a data segment for the first pathological episode and for subsequent pathological episodes. If it becomes desirable to use memory space where such information is being stored, in some examples the pathology detection circuit 410 overwrites the markers and the segments of the physiologic sensor signal from the subsequent episodes before overwriting the markers and the segment of the physiologic sensor signal from the first episode.

FIG. 5 is a block diagram of portions of a system 500 that provides for enhanced reporting of pathological episodes. The system 500 includes an IMD 502 and an external device 504. The IMD 502 includes an implantable sensor 505, a pathology detection circuit 510, and a communication circuit 530. The implantable sensor 505 produces a time-varying physiologic sensor signal of a subject. As described previously, the pathology detection circuit 510 detects a first pathological episode using the sensed physiologic sensor signal, deems that the first pathological episode has ended, detects at least one second pathological episode using the sensed physiologic sensor signal, and indicates the first and second pathological episodes as one pathological episode if the first and second episode are within a specified time interval.

The communication circuit 535 communicates information wirelessly with the external device 504. The pathology detection circuit 510 is configured to communicate an indication of the one episode of tachyarrhythmia to the external device 504. In certain examples, the indication includes a flag value indicating one or more pathological episodes occurred. In some examples, the indication includes a count of the number of sub-episodes in one episode group. In certain examples, the count is tracked in the external device. In some examples, the indication includes a sampled segment of a physiologic sensor signal that indicates the pathology.

In some examples, the external device 504 includes an IMD programmer. In some examples, the external device 504 communicates with the IMD 502 via a third device (e.g., a repeater) that relays the communications between the IMD 502 and the external device 504. In some examples, the external device 504 is part of an advanced patient management (APM) system, and includes a server connected to a computer network such as the internet for example.

In some examples, the external device 504 includes a communication circuit 535 to communicate wirelessly with the IMD 502, a user interface 540, and a processor 545 communicatively coupled to the user interface 540 and the communication circuit 535. The user interface 540 may include one or more of a keyboard or key pad, a mouse, and a display screen.

The processor 545 may be a microprocessor and executes instructions in one or both of software and firmware to perform the functions described. The processor 545 displays the first and second pathological episodes as one episode or multiple pathological episodes according to input received via a user interface 540 at the external device 504. The IMD 502 may transmit representations of the first and second signal segments to the external device 504. In some examples, the external device 504 displays one or both of markers and segments of the physiologic sensor signal for the grouped episode or for the multiple episodes.

In some examples, the processor 545 displays the grouped multiple pathological episodes using multiple segments of a sensed physiologic sensor signal when prompted to do so according to user input received via the user interface. For instance, the default setting in the external device 504 may be to display episodes indicated as grouped by the IMD 502 as one episode of a detected pathology. In certain examples, a color code can be used on the display to differentiate single episode segments from the multiple sub-episode segments. The grouped episode can be displayed as the multiple sub-episode segments in response to user input such as a mouse click on the grouped episode. This allows more detail about the grouped episodes to be displayed when requested by a clinician.

In some examples, the external device 504 displays statistics related to each pathological episode in association with a physiologic sensor signal segment of each episode according to information received from the IMD 502. The statistics may include a duration of each pathological episode, or may include a start and end time of the pathological episodes. In certain examples, the statistics include the detection parameters used to detect a pathological episode. In certain examples, the external device 504 displays statistics related to the detected pathology as one episode. In certain examples, the external device displays arrhythmia statistics related to each of the multiple segments of multiple sub-episodes.

In some examples, the statistics include a signal morphology of each pathological episode. In certain examples, the statistics include a calculation of a regularity of the morphology. For instance, the regularity may be a regularity of the timing of a feature evident in the physiologic sensor signal segments, or conversely a measure of variability of the feature. Examples of a measure of variability include, among other things, a variance of a marker representing the occurrence of the feature in the signal segment or a standard deviation of the marker.

If the pathology includes arrhythmia, the statistics may include a depolarization rate of an arrhythmia episode. In certain examples, the statistics include timing relationships between the atrial and ventricular depolarizations. In some examples, the statistics include a calculation of rate stability for each episode of arrhythmia. In some examples, the statistics include an indication of whether the IMD 502 provided a therapy in response to detecting the arrhythmia. In some examples, the statistics include an indication of whether the IMD 502 switched operating modes in response to detecting the arrhythmia (e.g., a switch from a DDD pacing mode to a VVI or VOO pacing mode). In certain examples, the statistics includes the number of times the IMD 502 switched operating modes during a grouped episode.

According to some examples, some of the functions described as being performed by the IMD 502 can be performed by the external device 504. For instance, the IMD 502 may communicate one or more statistics related to multiple pathological episodes to the external device 504. The external device 504 may then determine whether the episodes occurred within a specified time interval and group the pathological episodes accordingly. The time interval may be programmed into the external device 504. In some examples the IMD 502 transmits, for each detected pathological episode, one or both of a timestamp and an indication of the type of episode to the external device. The external device 504 uses the timestamps to determine whether multiple episodes should be indicated as being one pathological episode to the user.

In some examples, the IMD 502 communicates criteria additional to the timestamp information to the external device 504 for the external device 504 to use in grouping pathological episodes. In certain examples, the additional criteria may include segments of one or more physiological signals, and the external device 504 determines a similarity of morphology of the physiologic sensor signal segments of the multiple episodes.

In certain examples, the additional criteria include a depolarization rate determined by the IMD 502 or an assessment by the IMD 502 of heart rhythm stability. In certain examples, the additional criteria may include using an event, indicated by the IMD 502 to have been sensed in one heart chamber, to group events indicated by the IMD 502 to have been sensed in another heart chamber as one episode. In certain examples, the additional criteria include one or both of the similarity of IMD-determined posture of the subject during the episodes or the similarity of IMD-determined activity of the subject during the episodes. In certain examples, the additional criteria include information obtained from a second sensor. The second sensor may be included in the IMD 502 or separate from the IMD 502.

As described previously, the external device 504 may be part of an APM system. In some examples, the external device 504 uses the APM system to notify a clinician of a certain event reflected in the statistics. The clinician can choose a trigger point for being notified via the user interface 540 (e.g., when the patient has a tachyarrhythmia episode that lasts longer than a specified time). The statistic defining such a trigger point is programmable depending on the clinician's needs, the pathology or pathologies of concern, and the patient's physical condition. The grouping of pathological episodes may make it easier for a clinician to interpret signals sensed by physiologic sensor.

### Additional Notes

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An apparatus comprising:
a sensor circuit configured to produce a time-varying physiologic sensor signal of a subject; and
a pathology detection circuit communicatively coupled to the sensor and configured to:
detect a first pathological episode using the physiologic sensor signal;
deem that the first pathological episode has ended;
detect at least one second pathological episode using the physiologic sensor signal; and
indicate the first and second pathological episodes as one pathological episode if the first and second episodes are detected within a specified time interval.

2. The apparatus of claim 1:
wherein the sensor circuit includes an implantable cardiac signal sensing circuit configured to produce a sensed cardiac signal representative of cardiac activity of a subject; and
wherein the pathology detection circuit is configured to:
detect a first episode of arrhythmia using the sensed cardiac signal;
deem that the first episode of arrhythmia has ended;
detect at least one second episode of arrhythmia using the sensed cardiac signal; and
indicate the first and second episodes of arrhythmia as one episode of arrhythmia if the first and second episodes are detected within the specified time interval.

3. The apparatus of claim 2, wherein the pathology detection circuit is configured to adjust at least one arrhythmia detection parameter when one or more arrhythmia episodes are detected within the specified time interval of a previous arrhythmia episode.

4. The apparatus of claim 3, wherein, when multiple tachyarrhythmia episodes are detected within the specified time interval, the pathology detection circuit is configured to, at least one of:
reduce an atrial tachyarrhythmia response entry count;
increase an atrial tachyarrhythmia response exit count; or
reduce a tachyarrhythmia detection rate zone threshold.

5. The apparatus of claim 2, wherein the pathology detection circuit is configured to:
determine whether the first and second episodes are one episode of arrhythmia by applying an additional detection criterion to the first and second arrhythmia episodes when the second episode is detected within the specified time interval; and
indicate the first and second episodes of arrhythmia as one episode of arrhythmia according to the additional detection criterion.

6. The apparatus of claim 5, wherein the pathology detection circuit is configured to obtain, as the additional detection criteria, at least one of:
similarity of heart rate among detected episodes;
an assessment of heart rate stability; or
a morphology analysis of the detected arrhythmia.

7. The apparatus of claim 5, including:
a second implantable sensor circuit configured to produce a second sensor signal representative of hemodynamic function of the heart, and
wherein the pathology detection circuit is configured to determine whether the first and second episodes are one episode of arrhythmia according to the second sensor signal.

8. The apparatus of claim 1, wherein the sensor circuit includes at least one of:
an implantable respiration sensor configured to produce a sensed respiration signal representative of respiration activity of the subject, wherein the first and second pathological episodes include episodes of fast respiration rate;
an implantable blood pressure sensor configured to produce a sensed pressure signal representative of blood pressure of the subject, wherein the first and second pathological episodes include episodes of one or more low blood pressure and high blood pressure;
an implantable blood gas sensor configured to produce a sensor signal associated with changes in the fluid oxygen saturation of blood, wherein the first and second pathological episodes include episodes of a low level of oxygen saturation in blood;
an implantable chemical sensor configured to produce a sensor signal associated with changes in the blood pH, wherein the first and second pathological episodes include episodes of a change in blood pH that exceeds a specified change value; or
a heart sound sensor configured to produce a heart sound signal associated with mechanical activity of a patient's heart, wherein the first and second pathological episodes include episodes of a change in a measured heart sound parameter that exceeds a specified change value.

9. The apparatus of claim 8, wherein the pathology detection circuit is configured to indicate the first pathological episode and the second pathological episode as one pathological episode according to additional detection criteria, wherein the additional detection criteria includes at least one of:
similarity of morphology of the physiologic sensor signal during the detected episodes;
similarity of device-determined posture of the subject during the episodes; or
similarity of device-determined activity level of the subject during the episodes.

10. The apparatus of claim 1, wherein the pathology detection circuit includes a pathological episode counter, and
wherein the pathology detection circuit is configured to:
update the pathological episode counter upon detecting the first pathological episode;
maintain an episode count of the pathological episode counter when one or more later pathological episodes are detected within the specified time interval of a previous pathological episode; and
update the pathological episode counter upon detecting a later pathological episode after the time interval expires.

11. A system comprising:
an implantable medical device (IMD) comprising:
an implantable sensor configured to produce a time-varying physiologic sensor signal of a subject; and
a pathology detection circuit communicatively coupled to the sensor and configured to:
detect a first pathological episode using the physiologic sensor signal;
deem that the first pathological episode has ended;
detect at least one second pathological episode using the physiologic sensor signal; and
indicate the first and second pathological episodes as one pathological episode if the first and second episodes are detected within a specified time interval; and
a communication circuit communicatively coupled to the pathology detection circuit and configured to communicate information wirelessly with an external device, wherein the pathology detection circuit is configured to communicate an indication of the one pathological episode to the external device; and
the external device, comprising:
a user interface;
a communication circuit; and
a processor communicatively coupled to the user interface and the communication circuit and configured to display the first and second pathological episodes as one pathological episode according to an indication received from the IMD.

12. The system of claim 11, wherein the processor is configured to:
display the indicated one pathological episode as multiple pathological episodes according to input received via a user interface at the external device;
display the multiple pathological episodes using multiple segments of the physiologic sensor signal according to input received via the user interface; and
display a statistic related to a pathological episode in association with a segment of the physiologic sensor signal according to information received from the IMD.

13. The system of claim 11,
wherein the pathology detection circuit is configured to:
detect the first pathological episode using a first segment of the physiologic sensor signal;
detect the second pathologic episode using a second segment of the physiologic sensor signal;
transmit representations of the first and second segments to the external device; and
wherein the processor is configured to:
display a statistic related to the detected pathology as one episode; and
display a pathological statistic related to each of the first and second segments.

14. A method comprising:
detecting a first pathological episode of a subject with a medical device, wherein the first pathological episode is detected using a sensed time-varying physiologic sensor signal;
deeming, with the medical device, that the first pathological episode has ended;
detecting at least one second pathological episode with the medical device; and
deeming, with the medical device, that the first and second pathological episodes are one episode if the first and second episodes are detected within a specified time interval.

15. The method of claim 14,
wherein detecting a first pathological episode includes detecting a first episode of arrhythmia with an IMD;
wherein deeming that the first pathological episode has ended includes deeming that the first episode of arrhythmia has ended;
wherein detecting at least one second pathological episode includes detecting at least one second episode of arrhythmia with the IMD; and
wherein deeming that the first and second pathological episodes are one episode includes deeming that the first and second episodes of arrhythmia are one episode of arrhythmia if the first and second episodes are detected within the specified time interval.

16. The method of claim 15, including adjusting at least one arrhythmia detection parameter if multiple arrhythmia episodes are detected within the specified time interval of a previous arrhythmia episode.

17. The method of claim 16, wherein adjusting at least one arrhythmia detection parameter includes at least one of:
reducing an atrial tachyarrhythmia response entry count;
increasing an atrial tachyarrhythmia response exit count; or
reducing a tachyarrhythmia rate detection zone threshold.

18. The method of claim 15, including:
determining whether the first and second episodes are one episode of arrhythmia by applying an additional detection criterion to the first and second arrhythmia episodes when the second episode is detected within the specified time interval, and
wherein indicating the first and second episodes of arrhythmia as one episode of arrhythmia includes indicating the first and second episodes of arrhythmia as one episode of arrhythmia according to the additional detection criteria.

19. The method of claim 18, wherein the additional detection criteria includes at least one of:
an assessment of heart rhythm stability;
a morphology analysis of the detected arrhythmia; or
an assessment of hemodynamic stability.

20. The method of claim 14, wherein detecting the first and second pathological episodes with the medical device includes detecting at least one of:
first and second episodes of fast respiration rate;
first and second episodes of one or more of low blood pressure and high blood pressure;
first and second episodes of low levels of oxygen saturation in blood;
first and second episodes of a change in blood pH that exceeds a specified change value; or
first and second episodes of a change in a measured heart sound parameter that exceeds a specified change value.

21. The method of claim 20, wherein deeming that the first and second pathological episodes are one pathological episode includes deeming that the first and second pathological episodes are one pathological episode according to additional detection criteria, wherein the additional detection criteria includes at least one of:
similarity of morphology of the physiologic sensor signal during the detected first and second episodes;
similarity of device-determined posture of the subject during the first and second episodes; or
similarity of device-determined activity level of the subject during the first and second episodes.

22. The method of claim 14, including:
updating a pathological episode counter upon detecting the first pathological episode;
maintaining an episode count when one or more later pathological episodes are detected within the specified time interval of a previous pathological episode; and
updating the arrhythmia episode counter upon detecting a later pathological episode after the specified time interval expires.

23. The method of claim 14, wherein the medical device is an IMD, and wherein indicating the first and second pathological episodes as one pathological episode includes:
transmitting the indication from the IMD to an external device; and
displaying the first and second pathological episodes as one pathological episode or as multiple pathological episodes according to input received via a user interface at the external device.

24. The method of claim 23, including
displaying the multiple pathological episodes using multiple segments of the physiologic sensor signal when prompted to do so via the user interface; and
displaying statistics related to each pathological episode in association with the associated signal segment.

25. The method of claim 24, wherein displaying statistics includes displaying at least one of:
a sensed cardiac depolarization rate during each pathological episode;
a duration of each pathological episode; or
a signal morphology of the physiologic sensor signal during each pathological episode.
